(19) United States
(12) Patent Application Publication
(10) Pub. No.: US 2004/0142902 A1
(43) Pub. Date: Jul. 22, 2004

(54) Implant dosage form and use thereof for the delivery of a cholosterol lowering agent

(76) Inventor: Harry A.J. Struijker- Boudier, Rijckholt (NL)

Correspondence Address: DURECT CORPORATION, 10240 BUBB ROAD, CUPERTINO, CA, 95014, US

(21) Appl. No.: 10/432,644

(22) PCT Filed: Nov. 8, 2001
(86) PCT No.: PCT/US01/46714

(51) Int. Cl. A61K 31/7024; A61K 31/401; A61K 31/366; A61K 31/225

(52) U.S. Cl. 514/53; 514/423; 514/460; 514/548

(57) ABSTRACT

Implanted, sustained release dosage forms, devices and methods for the delivery of a cholesterol lowering agent.

This application takes priority from U.S. Provisional Application 60/249,644, filed 16 Nov., 2000.

### FIELD OF THE INVENTION

The invention relates to implanted devices for management of cholesterol levels.

### BACKGROUND OF THE INVENTION

Coronary heart disease (CHD) remains the leading cause of death in the industrialized countries. Despite recent declines in CHD mortality, CHD is still responsible for more than 500,000 deaths in the U.S. annually. It is estimated that CHD, directly and indirectly, costs the U.S. more than $100 billion a year.

A primary cause of CHD is atherosclerosis, a disease characterized by the deposition of lipids in the arterial vessel wall and a resulting narrowing of the vessel passages, and ultimately by a hardening of the vascular system. Atherosclerosis as manifested in its major clinical complication, ischemic heart disease, is thought to begin with local injury to the arterial endothelium, followed by proliferation of arterial smooth muscle cells from the medial layer to the intimal layer, which is accompanied by deposition of lipid and accumulation of foam cells in the lesion. As the atherosclerotic plaque develops, it progressively occludes more and more blood vessel and can eventually lead to ischemia or infarction.

Hypercholesterolemia is an important risk factor associated with CHD. For example, in December 1984, a National Institute of Health Consensus Development Conference Panel concluded that lowering plasma cholesterol levels (specifically blood levels of low-density lipoprotein cholesterol) will reduce the risk of heart attacks due to CHD. Elevated cholesterol levels are also associated with a number of disease states, including restenosis, angina, cerebral arteriosclerosis, and xanthoma.

Therapeutic agents for management of hypercholesterolemia include: (1) a hydroxy-methylglutaryl-CoA (HMG-CoA) reductase inhibitor which restrains synthesis of cholesterol, (2) probucol which mainly promotes catabolism from cholesterol to a bile acid and excretion thereof, (3) an anion exchange resin which mainly restrains absorption of cholesterol and promotes excretion of a bile acid (cholestyramine, for example), and (4) a clofibrate-type drug (clofibrate, for example). Currently, HMG-CoA reductase inhibitors are the most effective agents currently available for lowering plasma levels of low-density lipoprotein cholesterol (LDL-C) and are the mainstay therapy for hyperlipidemia. Several large; controlled clinical trials have confirmed significant reductions in rates of coronary heart disease morbidity and death with long-term statin therapy in patients with mild to severe hypercholesterolemia (see, e.g., Blumenthal RS Am Heart J 2000 April; 139(4): 577-83).

As is evident from the above, there is a great need for devices and methods for effective and practical management of cholesterol, particularly serum cholesterol levels, with better efficacy and reduced side effects. The present invention addresses this problem.

### SUMMARY OF THE INVENTION

The invention features devices and methods for the delivery of a cholesterol lowering agent (e.g., an inhibitor of endogenous cholesterol biosynthesis such as an HMG CoA reductase inhibitor) to reduce levels of serum cholesterol and/or cholesterol accumulation and deposition. In the present invention, a drug formulation comprising a cholesterol lowering agent is provided parenterally in a sustained release dosage form, e.g., as an injected matrix or stored within a drug delivery device.

The drug delivery device may be an implantable device such as an osmotic pump, an electrochemical pump, an electromechanical pump, an electroosmotic pump, a piezoelectric pump, an effervescent pump, a vapor pressure pump, an electrolytic pump, a hydrolytic system, an electrodiffusion system, an elastomeric system, an osmotic bursting matrix, a bioerodable implant, a sustained release injectable, a microparticulate suspension, a liposome formulation, a micelle formulation, an oil suspension, an encapsulated particulate suspension system, and microsphere system, an erosion-based system, or a depot.

Once released from the dosage form, the drug formulation enters the systemic circulation and is transported to the site of action in the body to modulate cholesterol levels, (e.g. the liver, heart, brain or other site of cholesterol synthesis or deposition).

Alternatively, in another embodiment, the dosage form maybe implanted or injected into a site in the body (i.e., implantation site) and a conduit, e.g. a catheter, can be used to transport the formulation from the dosage form for release at a site in the body distal from the implantation site, for example, the liver, brain, heart, etc.

In one aspect, the invention features methods of reducing cholesterol levels by delivery of a formulation comprising a cholesterol lowering agent to the subject from a sustained release dosage form. The formulation can be introduced to a subject via any parenteral delivery system with the ability to provide release of the formulation for a pre-selected period of time. In specific embodiments, the formulation is released at a low volume rate (e.g., from about 0.001 ml/day to 1 ml/day or from about 0.01 mg/day to 20 mg/day). Exemplary delivery methods include, but are not limited to, injectable sustained release dosage forms such as a depo-type preparations or an injectable formulation containing a sustained-release particulate preparation, e.g., microspheres or microcapsules.

In a particular aspect, the invention features devices for and methods of treating elevated cholesterol levels in a subject comprising the steps of implanting a drug delivery device within an implantation site in the body of a subject, where the drug delivery device is capable of drug release over a period of time. A formulation comprising a cholesterol lowering agent can be introduced from the device to a delivery site in an amount effective to reduce serum cholesterol levels and/or prevent accumulation of cholesterol and cholesterol by-products, e.g., atherosclerotic plaques. A long-term release formulation for use in such a device can be, e.g., contained in a reservoir or impregnated within a matrix within the drug delivery device.

A drug formulation comprising a cholesterol lowering agent is stored within a drug delivery device (e.g., contained in a reservoir or impregnated within a matrix within the controlled drug delivery device). The drug delivery device is implanted in the subject's body at an implantation site, and the drug formulation is released from the drug delivery device to a delivery site. The delivery site may be the same as, near, or distant from the implantation site.

Exemplary delivery methods and devices include, but are not limited to, injectable sustained release dosage forms including Sucrose Acetate Isobutyrate (SAIB), microspheres or microcapsules.). Non-injectable implants include preformed monolithic or coaxially extruded rods made of biodegradable polymer impregnated with drug. These rods may be prepared by melt extrusion or other techniques well known to those skilled in the art. Depots may include, for example, non-polymeric, biocompatible materials that can provide for release of drug over time. Exemplary non-polymeric materials include, but are not necessarily limited to, those described in U.S. Pat. Nos. 6,051,558; 5,747,058; and 5,968,542 (hereby expressly incorporated by reference). A depot may comprise a high viscosity liquid, such as a non-polymeric non-water-soluble liquid carrier material, e.g., SAIB or another compound such as a compound described in U.S. Pat. No. 5,747,058 (hereby expressly incorporated by reference).

There has been extensive research in the area of biodegradable controlled release systems for bioactive compounds. Biodegradable matrices for drug delivery are useful because they obviate the need to remove the drug-depleted device. The most common matrix materials for drug delivery are polymers. The field of biodegradable polymers has developed rapidly since the synthesis and biodegradability of polylactic acid was reported by Kulkarni et al., in 1966 (“Polylactic acid for surgical implants,” Arch. Surg., 93:839). Examples of other polymers which have been reported as useful as a matrix material for delivery devices include polyanhydrides, polyesters such as polyglycolides and polylactide-co-glycolides, polyamino acids such as polylysine, polymers and copolymers of polyethylene oxide, acrylic terminated polyethylene oxide, polyamides, polyurethanes, polyorthoesters, polyacrylonitriles, and polyphosphazenes. See, for example, U.S. Pat. Nos. 4,891,225 and 4,906,474 to Langer (polyanhydrides), U.S. Pat. No. 4,767,628 to Hutchinson (polylactide, polylactide-co-glycolide acid), and U.S. Pat. No. 4,530,840 to Tice, et al. (polylactide, polyglycolide, and copolymers).

Degradable materials of biological origin are well known, for example, crosslinked gelatin. Hyaluronic acid has been crosslinked and used as a degradable swelling polymer for biomedical applications (U.S. Pat. No. 4,957,744 to Della Valle et al.; (1991) “Surface modification of polymeric biomaterials for reduced thrombogenicity,” Polym. Mater. Sci. Eng., 62:731-735).

Biodegradable hydrogels have also been developed for use in controlled drug delivery as carriers of biologically active materials such as hormones, enzymes, antibiotics, antineoplastic agents, and cell suspensions. Temporary preservation of functional properties of a carried species, as well as the controlled release of the species into local tissues or systemic circulation, have been achieved. See for example, U.S. Pat. No. 5,149,543 to Cohen. Proper choice of hydrogel macromers can produce membranes with a range of permeability, pore sizes and degradation rates suitable for a variety of applications in surgery, medical diagnosis and treatment.

Many dispersion systems are currently in use as, or being explored for use as, carriers of substances, particularly biologically active compounds. Dispersion systems used for pharmaceutical and cosmetic formulations can be categorized as either suspensions or emulsions. Suspensions are defined as solid particles ranging in size from a few nanometers up to hundreds of microns, dispersed in a liquid medium using suspending agents. Solid particles include microspheres, microcapsules, and nanospheres. Emulsions are defined as dispersions of one liquid in another, stabilized by an interfacial film of emulsifiers such as surfactants and lipids. Emulsion formulations include water in oil and oil in water emulsions, multiple emulsions, microemulsions, microdroplets, and liposomes. Microdroplets are unilamellar phospholipid vesicles that consist of a spherical lipid layer with an oil phase inside, as defined in U.S. Pat. Nos. 4,622,219 and 4,725,442 issued to Haynes. Liposomes are phospholipid vesicles prepared by mixing water-insoluble polar lipids with an aqueous solution. The unfavorable entropy caused by mixing the insoluble lipid in the water produces a highly ordered assembly of concentric closed membranes of phospholipid with entrapped aqueous solution.

U.S. Pat. No. 4,938,763 to Dunn, et al., discloses a method for forming an implant in situ by dissolving a non-reactive, water insoluble thermoplastic polymer in a biocompatible, water soluble solvent to form a liquid, placing the liquid within the body, and allowing the solvent to dissipate to produce a solid implant. The polymer solution can be placed in the body via syringe. The implant can assume the shape of its surrounding cavity. In an alternative embodiment, the implant is formed from reactive, liquid oligomeric polymers which contain no solvent and which cure in place to form solids, usually with the addition of a curing catalyst.

The invention also may employ “microspheres” (also known as “microparticles” or nanospheres” or “nanoparticles”) which are small particles, typically prepared from a polymeric material and typically no greater in size than about 10 micrometers in diameter. For reference, please refer generally to “Encyclopedia of Controlled Drug Delivery” 1999, published by John Wiley & Sons Inc, edited by Edith Mathiowitz. For example, U.S. Pat. No. 6,291,013 discloses polylactic acid microspheres, prepared by emulstion techniques containing a physiologically active substance and having an average particle size of about 1 to 250 micrometers.

In another particular aspect, the invention features methods of treating a subject having elevated serum cholesterol levels by systemic delivery of a formulation comprising an inhibitor of cholesterol synthesis (e.g., an HMG CoA reductase inhibitor) to the subject via an implantable drug delivery device, where such formulation is delivered at a rate and/or concentration sufficient to lower cholesterol in a subject. In specific embodiments, the formulation comprises a statin such as cerivastatin, which can be administered at a rate of from about 0.1 μg per hour to 200 μg per hour for a period of at least a week, and can be delivered for a period of at least about a month, and or at least about six months.

In another aspect, the invention features local administration of a cholesterol biosynthesis inhibitor to suppress cholesterol production, deposition and/or the accumulation in a specific region, e.g., near the liver, heart or brain.

In various exemplary embodiments of the invention and various aspects thereof, drug of the drug formulation administered is delivered at a low dose rate due the potency of the subject drugs, e.g., from about 0.01 μg/hr or 0.1 μg/hr, 0.25 μg/hr, 1 μg/hr, generally up to about 200 μg/hr. Specific ranges of amount of drug delivered will vary depending upon, for example, the potency and other properties of the drug used and the therapeutic requirements of the subject. In one specific embodiment, the formulation comprises a statin and, in a specific embodiment, is delivered at a rate of from about 0.01 μg/hr or 0.1 μg/hr, 0.25 μg/hr, 1 μg/hr, generally up to about 200 μg/hr. In another exemplary embodiment, the drug formulation is delivered at a low volume rate e.g., a volume rate of from about 0.001 ml/day to about 1 ml/day.

A primary object of the invention is provide a method for convenient, long-term management of cholesterol production.

One advantage of the invention is that the devices and methods described herein provide effective management of cholesterol levels by administration of a relatively small quantity of a cholesterol lowering agent (e.g., an HMG CoA reductase inhibitor such as a statin). Given the long-term, chronic effects of cholesterol production, esterification, and/or deposition, this advantage is of considerable benefit for relatively long term (e.g., 1-4 months) dosage regimes. Furthermore, the method may be more cost-effective than current prescription drugs, and thus may make cholesterol management available to a broader population.

Another advantage of the invention is that the cholesterol lowering agent can be administered to provide for a substantially constant lowered cholesterol levels. In contrast, oral delivery of these agents provides for intermittent lowering of cholesterol levels, a product of underdosing inherently associated with bolus administration.

The present invention is also advantageous in that it can provide for safe, effective therapy while minimizing the risk of undesirable side effects.

Another advantage of the invention is that the invention can be used to deliver relatively small quantities of cholesterol lowering agents accurately and precisely. Thus, the invention allows for the convenient use of these drugs for treatment, and particularly for the delivery of small amounts locally, e.g., to control the production of β-amyloid production in the brain.

Another notable advantage of the invention is that the implanted device increases patient compliance with a prescribed therapeutic regimen. This is particularly important since compliance is particularly difficult to achieve in prophylactic treatment before the onset of disease or symptom and since the population that needs treatment often has difficulty with compliance, e.g., the infirmed, the elderly and/or people with neurological disorders. Improved compliance will provide an improved therapeutic outcome in the patient.

A further advantage is that a therapeutically effective dose of a cholesterol lowering agent can be delivered at such relatively low volume rates, e.g., from about 0.001 ml/day to 1 ml/day so as to minimize tissue disturbance or trauma near the site where the formulation is released. The formulation may be released at a rate of, for example, 0.01 micrograms per day up to about 20 milligrams per day. Dosage depends on a number of factors such as potency, bioavailability, and toxicity.

Another advantage of the invention is that substantially continuous delivery of small quantities of cholesterol lowering agent (e.g., a HMG CoA reductase inhibitor such as a statin) is effective in long-term (e.g., chronic) administration (e.g., from several weeks or from about 1 to 12 months or more).

Yet another advantage is that the invention provides for precise delivery of the selected cholesterol lowering agent, thus allowing delivery of lower doses and/or for delivery of precisely metered doses at consistent delivery volume rates (e.g. on the order of microliters to milliliters per hour).

These and other objects, advantages and features of the present invention will become apparent to those persons skilled in the art upon reading the details of the methodology and compositions as more fully set forth below.

### BRIEF DESCRIPTION OF THE DRAWINGS

FIG. 1 illustrates systemic delivery of the drug formulation using an implanted drug delivery device.

FIG. 2 is a cut-away view of an exemplary drug delivery device useful in the present invention.

FIG. 3 is a cut-away view of an exemplary drug delivery device comprising a catheter.

### DESCRIPTION OF THE PREFERRED EMBODIMENTS

The invention is not limited to the specific methodology, devices, therapeutic formulations, and syndromes described. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “and”, and “the” include plural referents unless the context clearly dictates otherwise. Thus, for example, reference to “a drug delivery device” includes a plurality of such devices and reference to “the method of delivery” includes reference to equivalent steps and methods known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the compositions and methodologies which are described in the publications which might be used in connection with the presently described invention. The publications discussed herein are provided solely for their disclosure prior to the filing date of the present application. Nothing herein is to be construed as an admission that the invention is not entitled to antedate such a disclosure by virtue of prior invention.

Definitions

The terms “reduced cholesterol levels” and “lowered cholesterol levels” as used interchangeably herein are intended to encompass a reduction in serum cholesterol, reduction in cholesterol accumulation and/or deposition, and a reduction of cholesterol by-products, i.e. products associated with elevated cholesterol levels such as amyloid plaques. Generally, reduced cholesterol levels are between, for example, 50-95% of the levels in the untreated subject, or between 70-85% of the levels, preferably between 60-85% of the levels, in the subject prior to treatment.

The term “cholesterol lowering agent” as used herein is generally meant to refer to compounds which reduce the level of serum cholesterol, reduce cholesterol accumulation and deposition, and/or reduce the production of by-products of cholesterol (e.g. amyloid plaques). These agents may function by a variety of mechanisms and include compounds which increase uptake of cholesterol by the liver, compounds which block endogenous cholesterol biosynthesis, compounds which prevent uptake of dietary cholesterol, compounds which enhance clearance of cholesterol from the body, and the like. Use of the term “cholesterol lowering agent” is not meant to be limiting to use of, or formulations comprising, only one of these selected compounds. Furthermore, reference to a selected specific compound, e.g., reference to “a statin,” is understood to be only exemplary of the drugs suitable for delivery according to the methods of the invention, and is not meant to be limiting in any way. The term is also meant to encompass compounds that specifically decrease LDL and/or alter the LDL:HDL ratio, i.e. that reduce the level of the unwanted form of cholesterol without actually reducing the overall serum cholesterol levels. Additional exemplary cholesterol lowering agents include, but are not necessarily limited to hypolipidemic agents (e.g., nicotinic acid, probucol, etc.), bile acid-binding resins (e.g., cholestyramine), and fibric acid derviatives (e.g., clofibrate).

The term “inhibitor of cholesterol biosynthesis” as used herein refers to a compound with the ability to inhibit an enzyme in a subject's endogenous cholesterol biosynthetic pathway. This includes any compound that inhibits an enzyme involved in the biosynthetic pathway from the staring product 3-hydroxy-3-methylglutaryl coenzyme A (HMG CoA) to the production of cholesterol. Examples of agents that inhibit cholesterol biosynthesis by disrupting the cholesterol biosynthetic pathway include but are not limited to HMG CoA reductase inhibitors, HMG CoA synthase inhibitors, squalene synthase inhibitors, and squalene epoxidase inhibitors. In a particular embodiment, the inhibitor of biosynthesis is an HMG CoA reductase inhibitor, and more particularly the drug is a statin, e.g., lovastatin, cerivastatin, fluvastatin, pravastatin, simvaststin, etc.

The term “drug delivery device” refers to any means for containing and releasing a drug wherein the drug is released into a subject. The means for containment is not limited to containment in a walled vessel, but may be any type of containment device, including non-injectable devices (pumps etc) and injectable devices, including a gel, a viscous or semi-solid material or even a liquid. Drug delivery devices are split into five major groups: inhaled, oral, transdermal, parenteral and suppository. Inhaled devices include gaseous, misting, emulsifying and nebulizing bronchial (including nasal) inhalers; oral includes mostly pills; whereas transdermal includes mostly patches. Parenteral includes two sub-groups: injectable and non-injectable devices. Non-injectable devices are generally referred to as “implants” or “non-injectable implants” and include e.g., pumps and solid biodegradable polymers. Injectable devices are split into bolus injections, that are injected and dissipate, releasing a drug all at once, and depots, that remain discrete at the site of injection, releasing drug over time. Depots include e.g., oils, gels, liquid polymers and non-polymers, and microspheres. Many drug delivery devices are described in Encyclopedia of Controlled Drug Delivery (1999), Edith Mathiowitz (Ed.), John Wiley & Sons, Inc.

The term “drug” as used herein, refers to any substance meant to alter animal physiology.

The term “dosage form” refers to a drug plus a drug delivery device. 0.9

The term “formulation” means any drug together with a pharmaceutically acceptable excipient or carrier such as a solvent such as water, phosphate buffered saline or other acceptable substance. A formulation may include one or more cholesterol lowering agents, for example, a two or more cholesterol lowering agents that are HMG CoA reductase inhibitors. An inhibitor of cholesterol biosynthesis can be combined with an additional ingredient that increases cholesterol metabolism, e.g., probucol. A formulation may have an active agent that mediates a separate biological response (e.g., an anticoagulant). A formulation may also encompass one or more carrier materials such as SAIB or other carrier materials such as described in U.S. Pat. Nos. 5,747,058 and 5,968,542.

The term “subject” is meant any subject, generally a mammal (e.g., human, canine, feline, equine, bovine, ursine, lepine, lupine, bufine, porcine, ungulate etc).

The term “systemic delivery” means delivery which permits drug to enter into the systemic circulation, e.g., intravenous, intra-arterial, intramuscular, subcutaneous, intra-adipose tissue, intra-lymphatic, etc.

The term “therapeutically effective amount” means an amount sufficient to bring about a desired physiological effect (e.g., a decrease in serum cholesterol levels and/or cholesterol deposition).

“Delivery site” as used herein is meant to refer to an area of the body to which drug is released from the dosage form, e.g., subcutaneous, intravenous, intra-arterial, intramuscular, intra-adipose tissue, and intra-lymphatic sites.

The term “implantation site” is used to refer to a site within the body of a subject at which a dosage form is introduced and positioned.

“Patterned” or “temporal” as used in the context of drug delivery means delivery of drug in a pattern, over a pre-selected period of time (e.g., other than a period associated with, for example a bolus administration, encompasses delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “substantially continuous” means delivery of drug (e.g., a statin) in a manner that is substantially uninterrupted for a pre-selected period of drug delivery.

The term “sustained release dosage form” is meant to refer to a drug dosage form that is capable of release of a drug formulation (e.g., a statin) over a pre-selected period of time rather than at one time as in a bolus administration.

The term “treatment” and the like refers to obtaining a desired pharmacologic and/or physiologic effect. The effect may be prophylactic in terms of completely or partially preventing a condition or symptom thereof or may be therapeutic in terms of a partial or complete cure for, relief from, or suppression of a disease. Treatment includes: (a) Preventing or diminishing the incidence of elevated cholesterol levels in a subject that may be predisposed but is not at the time displaying such elevated levels; (b) Reducing endogenous production of cholesterol; (c) reducing uptake of dietary cholesterol; (d) Inhibiting accumulation and deposition of cholesterol; and (e) Causing regression and/or amelioration in a subject with a disease or condition associated with elevated cholesterol levels.

Indications for Administration of Cholesterol Lowering Formulations

In general, administration of a formulation comprising a cholesterol lowering agent according to the invention can be used to facilitate management of elevated cholesterol levels associated with any of a wide variety of risk factors, disorders, conditions, or diseases. Conditions amenable to alleviation include, but are not necessarily limited to, diseases involving elevated serum cholesterol such as hypercholesterolemia; diseases involving cholesterol esterification and/or deposition, such as atherosclerosis; and diseases involving cholesterol-induced plaques, such as β-amyloid-associated neurological disorders.

Specific examples of conditions, diseases, disorders, and risk factors associated with elevated cholesterol production according to the present invention include, but are not necessarily limited to cardiovascular disease including atherosclerosis of coronary arteries and myocardial infarctions; cerebrovascular disease including atherosclerosis of the intracranial and/or extracranial arteries, stroke, and transient ischemic attacks; and disease involving cholesterol-associated plaque formation, e.g., Alzheimer's disease. The methods of the invention can be used to treat a subject that has displayed the symptoms of and/or been diagnosed with one or more of such conditions. The methods of the invention can also be used prophylactically to treat a subject at risk of a condition, e.g., a coronary and/or cerebrovascular event. Such risk factors include, but are not limited to, hypercholesterolemia, coronary artery disease (CAD), family history of coronary artery disease, hypertension, diabetes, cigarette smoking, and cerebrovascular disease. For example, where the risk factor is hypercholesterolemia, the serum total cholesterol concentrations of a subject are generally at least 5.2 mmol/liter (at least 200 mg/dl).

Cholesterol Lowering Agents and Formulations

The present invention provides methods for reducing cholesterol levels in a subject by long-term administration of a cholesterol lowering agent.

In one embodiment, the cholesterol lowering agent is an inhibitor of cholesterol biosynthesis, e.g., an inhibitor of HMG-CoA reductase enzyme. The primary rate limiting enzyme in the pathway is HMG CoA reductase, and thus cholesterol lowering agents of a specific embodiment regulate HMG CoA reductase at the level of transcription, translation, degradation, and/or at the switch from an inactive HMG CoA reductase to an active form.

In a preferred embodiment, the cholesterol lowering agents are HMG CoA reductase inhibitors of the statin family. These agents are described in detail, for example, mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, simvastatin and related compounds as disclosed in U.S. Pat. Nos. 4,448,784 and 4,450,171; fluvastatin and related compounds as disclosed in U.S. Pat. No. 5,354,772; atorvastatin and related compounds as disclosed in U.S. Pat. Nos. 4,681,893, 5,273,995 and 5,969,156; and cerivastatin and related compounds as disclosed in U.S. Pat. Nos. 5,006,530 and 5,177,080. Additional compounds are disclosed in U.S. Pat. Nos. 5,208,258, 5,130,306, 5,116,870, 5,049,696, RE 36,481, and RE 36,520. The lipophilicity of certain statins make them particularly suitable for subcutaneous delivery.

Other HMG CoA reductase inhibitors which may be employed herein include, but are not limited to, pyrazole analogs of mevalonolactone derivatives as disclosed in U.S. Pat. No. 4,613,610, indene analogs of mevalonolactone derivatives as disclosed in PCT application WO 86/03488, Trans-6-[2-(substitutedpyrrol-1-yl)alkyl]-pyran-2-ones and derivatives thereof as disclosed in U.S. Pat. No. 4,647,576, Searle's SC-45355 (a 3-substituted pentanedioic acid derivative) dichloroacetate, imidazole analogs of mevalonolactone as disclosed in PCT application WO 86/07054, 3-carboxy-2-hydroxy-propane-phosphonic acid derivatives as disclosed in French Patent No. 2,596,393, 2,3-di-substituted pyrrole, furan and thiophene derivatives as disclosed in European Patent Application No. 0221025, naphthyl analogs of mevalonolactone as disclosed in U.S. Pat. No. 4,686,237, octahydronaphthalenes such as disclosed in U.S. Pat. No. 4,499,289, keto analogs of mevinolin (lovastatin) as disclosed in European Patent Application No. 0,142,146 A2, as well as other known HMG CoA reductase inhibitors. In addition, phosphinic acid compounds useful in inhibiting HMG CoA reductase are disclosed in GB 2205837.

Agents which target other enzymes involved in cholesterol biosynthesis can also be used in the present methods. For example, squalene synthetase inhibitors suitable for use herein include, but are not limited to α-phosphonosulfonates disclosed in U.S. application Ser. No. 08/266,888, filed Jul. 5, 1994, now U.S. Pat. No. 5,712,396 (HX59b), those disclosed by Biller et al, J. Med. Chem. 1988, Vol. 31, No. 10, pp 1869-1871, including isoprenoid (phosphinylmethyl) phosphonates including the triacids thereof, triesters thereof and tripotassium and trisodium salts thereof as well as other squalene synthetase inhibitors disclosed in U.S. Pat. Nos. 4,871,721 and 4,924,024 and in Biller et al, J. Med. Chem., 1988, Vol. 31, No. 10, pp 1869 to 1871. In addition, other squalene synthetase inhibitors suitable for use herein include the terpenoid pyrophosphates disclosed by P. Ortiz de Montellano et al, J. Med. Chem.; 1977, 20, 243-249, the farnesyl diphosphate analog A and presqualene pyrophosphate (PSQ-PP) analogs as disclosed by Corey and Volante, J. Am. Chem. Soc. 1976, 98, 1291-1293, phosphinylphosphonates reported by McClard, R. W. et al, J.A.C.S., 1987, 109, 5544 and cyclopropanes.

Other cholesterol lowering agents mechanistically distinct from inhibitors of cholesterol biosynthesis that are suitable for use in the present methods include, but are not limited to, antihyperlipoproteinemic agents such as fibric acid derivatives, e.g., fenofibrate, gemfibrozil, clofibrate, bezafibrate, ciprofibrate, clinofibrate and the like, probucol and related compounds as disclosed in U.S. Pat. No. 3,674,836. Probucol and the fibrates increase the metabolism of cholesterol-containing lipoproteins. Other compounds, including bile acid sequestrants such as cholestyramine, colestipol and DEAE-Sephadex (Secholex®, Polidexide®), lipostabil (Rhone-Poulenc), Eisai E-5050 (an N-substituted ethanolamine derivative), imanixil (HOE-402), tetrahydrolipstatin (THL), istigmastanylphosphorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid, acipimox, acifran, neomycin, p-aminosalicylic acid, aspirin, poly(diallylmethylamine) derivatives such as disclosed in U.S. Pat. No. 4,759,923, quaternary amine poly (diallyldimethylammonium chloride) and ionenes such as disclosed in U.S. Pat. No. 4,027,009, and other known serum cholesterol lowering agents.

Two or more cholesterol lowering agents having either the same mechanism (e.g. two agents that inhibit HMG CoA reductase) or two different mechanisms (e.g., one agent that inhibits HMG CoA reductase and another agent which inhibits uptake of dietary cholesterol) can be used in a single formulation. For example, an inhibitor of cholesterol biosynthesis (e.g., a statin) can be combined with an additional ingredients including, but not limited to, farnesyl ester and ether compounds, probucol, fibric acids, clofibrate, niacin, gemfibrozol, LDL-receptor gene inducers, and zaragozic acid. Formulations of the invention may also comprise at least one cholesterol lowering agent and another active agent, i.e. an active agent that mediates a separate biological response (e.g., an anticoagulant).

A newer cholesterol lowering agent that may be used with the invention is rosuvastatin calcium.

A cholesterol lowering agent can be provided in any of a variety of formulations compatible with parenteral delivery, provided that such formulation is stable (i.e., not subject to degradation to an unacceptable amount at body temperature). The concentration of cholesterol lowering agent in the formulation may vary from about 0.1 wt. % to about 50 or 75 wt. %. The agent can be provided in any form suitable to be carried by the sustained release dosage from and released parenterally for systemic distribution, e.g., solid, semi-solid, gel, liquid, suspension, emulsion, osmotic dosage formulation, diffusion dosage formulation, erodible formulation, etc.

Formulations of the invention comprise a cholesterol lowering agent in a concentration of at least about 0.1 mg/mL, 0.5 mg/mL, 1 mg/mL, 10 mg/mL, 25 mg/mL, 50 mg/mL, 75 mg/mL, 100 mg/mL, 150 mg/mL, 200 mg/mL, 225 mg/mL, 250 mg/mL, 300 mg/mL, 350 mg/mL, 400 mg/mL, 450 mg/mL, 500 mg/mL, or greater. Formulations of the invention comprising cholesterol lowering agent are preferably in solution, e.g. are dissolved in a liquid.

Pharmaceutical grade organic or inorganic carriers and/or diluents suitable for parenteral delivery can be included in the formulations suitable for delivery according to the invention. Such physiologically acceptable carriers are well known in the art. Exemplary liquid carriers for use in accordance with the present invention can be sterile non-aqueous or aqueous solutions which contain no materials other than the active ingredient. The formulations can optionally further comprise a buffer such as sodium phosphate at physiological pH value, physiological saline or both (i.e., phosphate-buffered saline). Suitable aqueous carriers may optionally further comprise more than one buffer salt, as well as other salts (such as sodium and potassium chlorides) and/or other solutes.

In some exemplary embodiments, the cholesterol lowering agent is present in the formulation in a concentration of from about 0.1 mg/mL, 0.5 mg/mL to about 500 mg/mL, from about 1 mg/mL to about 450 mg/mL, from about 50 mg/mL to about 400 mg/mL, from about 75 mg/mL to about 300 mg/mL, or from about 100 mg/mL to about 250 mg/mL. Suitable low molecular weight alcohols include those which are pharmaceutically acceptable, and which can comprise an aromatic moiety, and which are relatively immiscible in water (e.g., less than about 5, less than about 4, less than about 3, less than about 2, less than about 1 gram can dissolve in 25 ml H₂O), including, but not limited to, benzyl alcohol, and derivatives thereof. Small amounts of other pharmaceutically acceptable substances such as other pharmaceutically acceptable alcohols, e.g., ethanol, or water, may also be present, and, if present, are present in an amount of less than about 10%, less than about 5%, or less than about 1%.

Formulations of particular interest for delivery are characterized in that the cholesterol lowering agent is present in a high concentration, as described above. The cholesterol lowering agent may be provided to the subject as a solution, a suspension, and/or a precipitate.

Suitable excipients can comprise dextrose, glycerol, alcohol (e.g., ethanol), and the like, and combinations of one or more thereof with vegetable oils, propylene glycol, polyethylene glycol, benzyl alcohol, benzyl benzoate, dimethyl sulfoxide (DMSO), organics, and the like to provide a suitable composition. In addition, if desired, the composition can comprise hydrophobic or aqueous surfactants, dispersing agents, wetting or emulsifying agents, isotonic agents, pH buffering agents, dissolution promoting agents, stabilizers, antiseptic agents and other typical auxiliary additives employed in the formulation of pharmaceutical preparations.

Of particular interest is a formulation in a depot form, such as a depot comprising sucrose acetate isobutyrate (SAIB). SAIB may be formulated with one or more suitable solvents which may be hydroxylic or nonhydroxylic and which may be used alone or in combination. Examples of solvents include ethanol, NMP, benzyl benzoate, benzoic acid, ethyl lactate, proplyene carbonate, glycofurol, and Miglyol 810, or mixtures thereof. The solvent can be added to SAIB in a ratio of from about 5 wt %-65 wt % solvent, usually 50 wt % or less The active agent, in a lypholized or dry powder form, may then be added to the SAIB/solvent mixture. The mixture is then mixed until homogeneous. The resulting mixture is then ready for parenteral injection.

A reduction in cholesterol in a subject may be measured using any technique that will be apparent to one skilled in the art upon reading the present disclosure. Such methods include, but are not limited to measurement of plasma cholesterol, measurement plasma triglycerides, measurement in plasma apolipoproteins, and measurement of HMG-CoA reductase activity in liver microsomes. Each of these can be either directly associated with or predictive of changes in cholesterol levels in a subject.

Implantation and Delivery Sites

The formulation can be introduced to a subject by injection or implantation at any suitable site using methods and devices well known in the art. Implantation sites include, but are not necessarily limited to a subdermal, subcutaneous, intramuscular, or other suitable site within a subject's body. Subcutaneous implantation sites are preferred because of convenience in implantation and, if necessary, removal of the drug dosage form. In some embodiments, the implantation site is at or near the delivery site (e.g., the delivery site is not distant from the implantation site). Exemplary subcutaneous delivery sites include external subcutaneous sites (e.g., under the skin of the arm, shoulder, neck, back, or leg) and internal subcutaneous sites within a body cavity (e.g., within the mouth). In addition, the delivery site can be the desired site of action (e.g., specific vessels at or near the heart or brain, etc.). In some embodiments, the delivery site is distant form the implantation site. Delivery of drug from a dosage form at an implantation site that is distant from a delivery site can be accomplished by providing the drug delivery device with a catheter, as described in more detail below.

An example of delivery and implantation for a SAIB depot formulation would be to inject a depot subcutaneously into the upper are of a subject using a needle and a standard syringe. Once the needle is withdrawn, the depot remains under the skin and becomes more viscous as hydrophilic solvent is released from the bulk of the hydrophobic matrix into surrounding tissue. From this stable location, the depot then releases the active at a relatively steady rate into the surrounding tissue, from where the drug finds its way into the circulatory system, and thence to its site of action. The depot may release the drug for weeks or months.

Delivery of Cholesterol Lowering Agents

Subjects suffering from or susceptible to high cholesterol levels and/or cholesterol deposition can receive prophylactic and/or therapeutic amounts of a cholesterol lowering agent according to the methods of the invention for any desired period of time. As elevated cholesterol levels and the conditions associated with such elevated levels are generally chronic, long-term administration is preferred (e.g.: continuous administration for at least 4 weeks) at, and the administration of a cholesterol lowering agent according to the invention can be sustained for several days (e.g., 2 to 5 days or more), to several weeks, months or years. Typically, delivery can be continued for a period ranging from about 1 week to about 1 month or about 12 months or more. The cholesterol lowering agent may be administered to an individual for a period of, for example, from about 20 days, from about 7 days or more, from about 10 days or more, from about 100 days or more, from about 1 week to about 4 weeks, from about 1 month to about 24 months, from about 2 months to about 12 months, from about 3 months to about 9 months, from about 1 month or more, from about 2 months or more, or from about 6 months or more; or other ranges of time, including incremental ranges, within these ranges, as needed.

Preferably, delivery of cholesterol lowering agent is substantially uninterrupted for a pre-selected period of drug delivery, and more preferably at a substantially constant, pre-selected rate or range of rates (e.g., amount of agent per unit time, or volume of drug formulation for a unit time). The agent can be delivered at a low volume rate of, for example, from about 0.001 μl/day or 0.04 μl/day to about 1 ml/day, usually from about 0.001 ml/day (1 μl/day) to at least about 500 μl/day or about 1 ml/day (i.e., from about 0.04 μl/hr to about 21 μl/hr to about 42 μl/hr), from about 2 μl/day to about 250 μl/day to 500 μl/day, from about 4 μl/day to about 100 μl/day, from about 5 μl/day to about 50 μl/day to 250 μl/day.

As many conditions and diseases associated with cholesterol are chronic, the methods of the present invention are particularly advantageous in providing long-term control and management of cholesterol levels in a subject. Sustained release dosage forms are convenient to the subject for long-term drug administration and can allow drug therapy to be conducted on an out-patient basis where the patient's health allows such. Implantable dosage forms, e.g., osmotic pumps and depots, have an added benefit in that they reduce the risk of infection associated with external pumps or other methods that require repeated breaking of the skin and/or maintenance of a port for administration.

Delivery of drug to a subcutaneous site at a low volume rate is a particularly preferred embodiment of the invention. In general, low volume rate drug delivery avoids accumulation of drug at the delivery site (e.g., depot or pooling effect) by providing for a rate of administration that is less than, the same as, or only very slightly greater than the rate of removal of drug from the delivery site (e.g., by absorption of drug in tissues at the site, movement of drug away from the site by flow of blood or other bodily fluids, etc.). Thus, in addition to providing an implantable system for long-term delivery of cholesterol lowering agents (e.g., a statin), the present invention also provides a method for treating chronic cholesterol level elevation by elegantly balancing the rates of drug absorption and drug delivery to accomplish administration of a therapeutically effective amount of drug, while avoiding accumulation of drug at the delivery site.

Subcutaneous delivery of a statin, the agent can be delivered at a rate of from about 0.01 μg/hr to about 200 μg/hr, usually from about 0.01 μg/hr, 0.25 μg/hr, or 3 μg/hr to about 85 μg/hr, and typically between about 5 μg/hr to about 100 μg/hr. In a specific exemplary embodiment, a statin is delivered at a rate of from about 0.01 μg/hr, 0.1 μg/hr, 0.25 μg/hr, 1 μg/hr, generally up to about 200 μg/hr. In another exemplary embodiment, the statin is delivered at a rate of from about 0.1 μg/hr to about 100 μg/hr, typically between about 1 μg/hr to about 100 μg/hr. Appropriate amounts of cholesterol lowering agent can be readily determined by the ordinarily skilled artisan based upon, for example, the relative potency of these drugs. The actual dose of drug delivered will vary with a variety of factors such as the potency and other properties of the selected drug used (e.g., lipophilicity, etc.).

Dosage Forms for use in the Invention

Any of a variety of parenteral dosage forms can be used in the present invention to accomplish delivery of a formulation according to the methods of the present invention. In general the drug release methods or dosage forms suitable for use in the invention are capable of retaining a quantity of drug formulation (e.g., contained in a drug reservoir or integrated into a substrate or matrix such as a polymer, binding solid, etc.) sufficient for treatment for a pre-selected period of sustained release. Exemplary dosage forms include pumps, depots, and implants. Drug delivery dosage forms that may be suitable for use with the present invention are described in Encyclopedia of Controlled Drug Delivery (1999), Edith Mathiowitz (Ed.), John Wiley & Sons, Inc.

The drug delivery device may deliver a formulation for several days e.g., at least 2 to at least 5 days or more, or from at least 1 month to at least 12 months or more, or from at least 10 days to at least 30 days to 100 days or more, from about 20 days to about 100 days or more; from about 2 week to about 4 weeks, from about 1 month to about 24 months, from about 2 months to about 12 months, from about 3 months to about 9 months, from about 1 month or more, from about 2 months or more, or from about 6 months or more; or other ranges of time, including incremental ranges, within these ranges, as needed. Release of drag from the device can be accomplished in any of a variety of ways according to methods well known in the art as discussed herein. Where the drug delivery device comprises a drug delivery catheter, drug can be delivered through the drug delivery catheter to the delivery site as a result of capillary action, as a result of pressure generated from the drug device, by diffusion, by electrodiffusion or by electroosmosis through the device and/or the catheter.

In general, the dosage form must be capable of carrying the drug formulation in such quantities and concentration as therapeutically required for treatment over the pre-selected period, and must provide sufficient protection to the formulation from degradation by body processes for the duration of treatment. For example, the dosage form can be surrounded by an exterior made of a material that has properties to protect against degradation from metabolic processes and the risk of, e.g., leakage, cracking, breakage, or distortion. This can prevent expelling of the dosage form contents in an uncontrolled manner under stresses it would be subjected to during use, e.g., due to physical forces exerted upon the drug release device as a result of movement by the subject or for example, in convective drug delivery devices, physical forces associated with pressure generated within the reservoir. The drug reservoir or other means for holding or containing the drug must also be of such material as to avoid unintended reactions with the active agent formulation, and is preferably biocompatible (e.g., where the dosage form is implanted, it is substantially non-reactive with respect to a subject's body or body fluids).

Suitable materials for the reservoir or drug holding means for use in the delivery devices of the invention are well known in the art. For example, the reservoir material may comprise a non-reactive polymer or a biocompatible metal or alloy. Suitable polymers include, but are not necessarily limited to, acrylonitrile polymers such as acrylonitrile-butadiene-styrene polymer, and the like; halogenated polymers such as polytetrafluoroethylene, polyurethane, polychlorotrifluoroethylene, copolymer tetrafluoroethylene and hexafluoropropylene; polyethylene vinylacetate (EVA), polyimide; polysulfone; polycarbonate; polyethylene; polypropylene; polyvinylchloride-acrylic copolymer; poly arbonate-acrylonitrile-butadien estyrene; polystyrene; cellulosic polymers; and the like. Further exemplary polymers are described in The Handbook of Common Polymers, Scott and Roff, CRC Press, Cleveland Rubber Co., Cleveland, Ohio.

Metallic materials suitable for use in the reservoir of the drug delivery devices include stainless steel, titanium, platinum, tantalum, gold and their alloys; gold-plated ferrous alloys; platinum-plated titanium, stainless steel, tantalum, gold and their alloys as well as other ferrous alloys; cobalt-chromium alloys; and titanium nitride-coated stainless steel, titanium, platinum, tantalum, gold, and their alloys.

Exemplary materials for use in polymeric matrices include, but are not necessarily limited to, biocompatible polymers, including biostable polymers and biodegradable polymers. Exemplary biostable polymers include, but are not necessarily limited to silicone, polyurethane, polyether urethane, polyether urethane urea, polyamide, polyacetal, polyester, poly ethylene-chlorotrifluoroethylene, polytetrafluoroethylene (PTFE or “Teflon™”), styrene butadiene rubber, polyethylene, polypropylene, polyphenylene oxide-polystyrene, poly-a-chloro-p-xylene, polymethylpentene, polysulfone and other related biostable polymers. Exemplary biodegradable polymers include, but are not necessarily limited to, polyanhydrides, cyclodextrtns, polylactic-glycolic acid, polyorthoesters, polycaprolactone, n-vinyl alcohol, polyethylene oxide/polyethylene terephthalate, polyglycolic acid, polylactic acid and other related bioabsorbable polymers.

Where the drug formulation is stored in a reservoir comprising metal or a metal alloy, particularly titanium or a titanium alloy having greater than 60%, often greater than 85% titanium is preferred for the most size-critical applications, for high payload capability and for long duration applications and for those applications where the formulation is sensitive to body chemistry at the implantation site or where the body is sensitive to the formulation. Most preferably, the drug delivery devices are designed for storage with drug at room temperature or higher.

Drug release devices suitable for use in the invention may be an osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc. In other embodiments, the drug release device comprises a high viscosity non-polymeric depot, such as SAIB, that may be injected under the skin or other site of parenteral administration.

Drug release devices based upon a mechanical or electromechanical infusion pump, can also be suitable for use with the present invention. Examples of such devices include those described in, for example, U.S. Pat. Nos. 4,692,147; 4,360,019; 4,487,603; 4,360,019; 4,725,852, and the like. In general, the present methods of drug delivery can be accomplished using any of a variety of refillable, non-exchangeable pump systems. Osmotic pumps are particularly preferred due to their combined advantages of more consistent controlled release and relatively small size. Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; 5,985,305; and the like.

Preferred osmotically-driven drug release systems are those that can provide for release of agent in a range of rates of from about 0.01 μg/hr to about 200 μg/hr, and which can be delivered at a volume rate range of, for example, from about 0.001 μl/day to about 100 μl/day (i.e., from about 0.0004 μl/hr to about 4 μl/hr), from about 0.04 μl/day to about 10 μl/day, from about 0.2 μl/day to about 5 μl/day, from about 0.5 μl/day to about 1 μl/day In general, in the present invention, the drug release system is selected to provide for delivery of a cholesterol lowering agent at a rate of from about 0.001 ml/day (1 μl/day) to at least about 500 μl/day or about 1 ml/day (i.e., from about 0.04 μl/hr to about 21 μl/hr to about 42 μl/hr), from about 2 μl/day to about 250 μl/day to 500 μl/day, from about 4 μl/day to about 100 μl/day, from about 5 μl/day to about 50 μl/day to 250 μl/day.

In one embodiment of particular interest, the volume/time delivery rate is substantially constant (e.g., delivery is generally at a rate ±about 5% to 10% of the cited volume over the cited time period). Delivery may be from about 0.1 μg/hr to about 200 μg/hr, and which can be delivered at a volume rate of from about 0.25 μl/day to about 100 μl/day (i.e., from about 0.0004 μl/hr to about 4 μl/hr), from about 0.04 μl/day to about 10 μl/day, and can be from about 0.2 μl/day to about 5 μl/day, or from about 0.5 μl/day to about 1 μl/day. In one embodiment, the volume/time delivery rate is substantially constant (e.g., delivery is generally at a rate ±about 5% to 10% of the cited volume over the cited time period).

The drug delivery dosage form can be a depot. Depots include injectable polymeric and non-polymeric biodegradable materials that may be high viscosity liquids. A depot may be subcutaneous. In one embodiment a depot comprises sucrose acetate isobutyrate (SAIB). SAIB may be formulated with one or more solvents such as glycofurol, ethanol or benzyl benzoate. Solvents may be nonhydroxylic, such as benzyl benzoate, NMP, DMSO or mixtures thereof, or it may be desirable to use a hydroxylic solvent such as ethanol, or glycerol. The solvent can be added to SAIB in a ratio of from about 5%-65% solvent, usually less than 50%. The active agent, for example a statin, in a lypholized or dry powder form, may then be added to the SAIB/solvent mixture. The mixture is then mixed until homogeneous. The resulting mixture is then ready for parenteral injection.

In other depot embodiments the dosage form includes microparticles or microspheres. Microparticles can be prepared by grinding to the appropriate particle size a mixture of biodegradable polymer and drug. The mixture may be prepared by a melt or solvent blend. Microspheres may be prepared by a number of methods familiar to those skilled in the art including spray drying, coacervation and emulsion techniques. For example, the methods described in U.S. Pat. No. 6,291,013 where a polymer solution containing drug is emulsified in water and then the solvent is removed by extraction, evaporation or a combination of the two may be used.

A biodegradable monolithic rod may also be used. An experimental example of such an embodiment, discussed in more detail below, is one in which a monolithic rod, wherein the rod contains 20% statin by weight within a polymer of 65:35 poly (DL-lactide-co-glycolide).

Delivery of a Formulation Using a Drug Delivery Device Comprising a Drug Delivery Catheter

In some embodiments wherein a drug delivery device is used, it may be desirable to provide a drug delivery catheter with the drug delivery device, e.g., where the implantation site and the desired delivery site are not the same or adjacent. The drug delivery catheter is generally a substantially hollow elongate member having a first end (or “proximal” end) associated with the drug release device of the drug delivery device, and a second end (or “distal”end) for delivery of the drug-comprising formulation to a desired delivery site. Where a drug delivery catheter is used, a first end of the drug delivery catheter is associated with or attached to the drug delivery device so that the lumen of the drug delivery catheter is in communication with the drug reservoir in the drug delivery device, so that a formulation contained in a drug reservoir can move into the drug delivery catheter, and out a delivery outlet of the catheter which is positioned at the desired delivery site.

The body of the catheter defines a lumen, which lumen is to have a diameter compatible with providing leak-proof delivery of drug formulation from the drug delivery device. Where the drug delivery device dispenses drug by convection, the size of the catheter lumen leading from the reservoir of the drug release system can be designed as described by Theeuwes (1975) J. Pharm. Sci. 64:1987-91.

The body of the catheter can be of any of a variety of dimensions and geometries (e.g., curved, substantially straight, tapered, etc.) that can be selected according to their suitability for the intended site for drug delivery. The distal end of the drug delivery catheter can provide a distinct opening for delivery of drug, or as a series of openings.

The drug delivery catheter may be produced from any of a variety of suitable materials, and may be manufactured from the same or different material as the reservoir of the drug release device. Impermeable materials suitable for use in production of the controlled drug release device as described above are generally suitable for use in the production of the drug delivery catheter. Exemplary materials from which the drug delivery catheter can be manufactured include, but are not necessarily limited to, polymers; metals; glasses; polyolefins (high density polyethylene (HDPE), low density polyethylene (LDPE), linear low density polyethylene (LLDPE), polypropylene (NP), and the like); nylons; polyethylene terephtholate; silicones; urethanes; liquid crystal polymers; PEBAX®; HYTREL®; TEFLON®; perflouroethylene (PFE) perflouroalkoxy resins (PFA); poly(methyl methacrylate) (PMMA); multilaminates of polymer, metals, and/or glass; nitinol; and the like.

The drug delivery catheter can comprise additional materials or agents (e.g., coatings on the external or internal catheter body surface(s)) to facilitate placement of the drug delivery catheter and/or to provide other desirable characteristics to the catheter. For example, the drug delivery catheter inner and/or outer walls can be coated with silver or otherwise coated or treated with antimicrobial agents, thus further reducing the risk of infection at the site of implantation and drug delivery.

In one embodiment, the drug delivery catheter is primed with a drug-comprising formulation, e.g., is substantially pre-filled with drug prior to implantation. Priming of the drug delivery catheter reduces delivery start-up time, i.e., time related to movement of the drug from the drug delivery device to the distal end of the drug delivery catheter. This feature is particularly advantageous in the present invention where the drug release device of the drug delivery device releases a cholesterol lowering agent at relatively low flow rates.

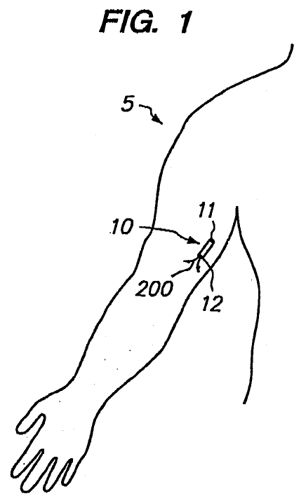

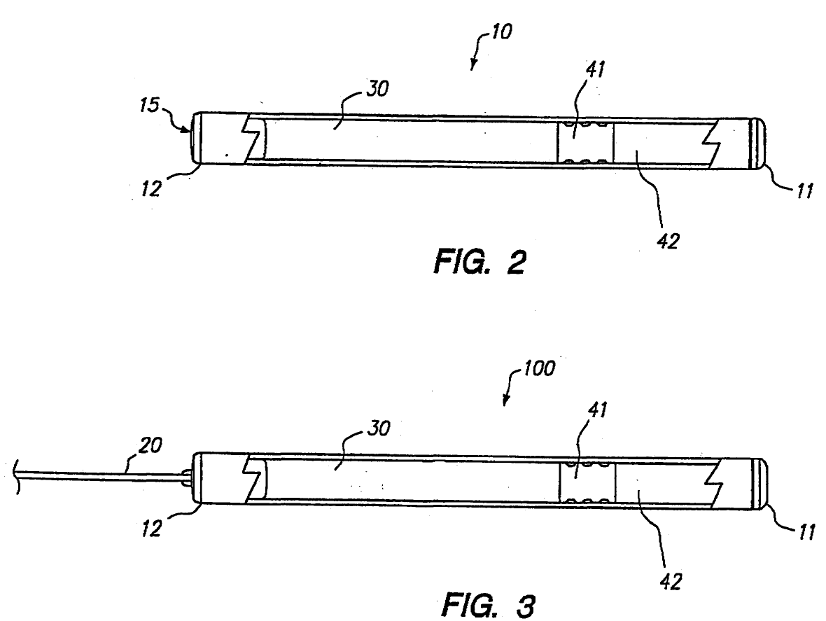

FIG. 1 illustrates one embodiment of the invention, wherein a formulation is delivered from an implanted drug delivery device that provides for sustained release of a formulation from a drug reservoir to a subcutaneous site. In this example, the drug delivery device 10 is implanted at a subcutaneous site in the patient's arm 5. Flow of drug from the device's drug reservoir and to the subcutaneous site is illustrated by arrows 200. FIG. 2 provides a perspective view of the exemplary drug delivery device 10 implanted in FIG. 1. The drug delivery device 10 comprises proximal and distal ends 11 and 12, with the distal end defining an orifice 15 through which drug exits the drug reservoir 30 for delivery to the subcutaneous site. In the exemplary device 10, controlled release of drug from the reservoir 30 is provided by an osmotic engine comprising a piston 41 and a chamber comprising an osmotic engine 42.

As shown in the cut-away of the drug delivery device in FIG. 3, the drug delivery system 100 comprises a drug delivery device 10 and a drug delivery catheter 20. The walls of the drug delivery catheter define a lumen, and the drug delivery catheter is associated with the drug delivery device 10 so that a drug delivery pathway is provided from the drug reservoir 30, through the orifice, and out the distal end 12 of the drug delivery device. The catheter 20 can be positioned for systemic delivery of drug, for example, subcutaneously.

Methods for implanting or otherwise positioning the dosage forms of the invention into the body are well known in the art. In general, placement of parenteral dosage forms will be accomplished using methods and tools that are well known in the art, and performed under aseptic conditions with at least some local or general anesthesia administered to the subject. Removal and/or replacement of the dosage forms, if necessary, can also be accomplished using tools and methods that are readily available.

Delivery of a Formulation Using a Depot

In one embodiment, the formulation is in the form of a depot, delivered and injected subcutaneously under the skin of the upper arm of a subject. In one example, a statin may be mixed with SAIB, which may be formulated with one or more solvents and which may be hydroxylic or nonhydroxylic. Examples of solvents include ethanol, NMP, benzyl benzoate; benzoic acid, ethyl lactate, propylene carbonate, glycofurol, and Miglyol 810 or mixtures thereof. The solvent can be added to SAIB in a ratio of from about 5 wt %-65 wt %, usually 50% solvent, or less. The active agent, for example a statin in a lypholized or dry powder form, may then be added to the SAIB/solvent mixture. The mixture is then mixed until homogeneous. The resulting mixture is then ready for parenteral injection.

Such a formulation may comprise, as an example, 1 g of cerivastatin which is then mixed with 9 g of a 85:15 mixture of SAIB and ethanol until a homogeneous mixture is achieved. Accurately weighed samples of the formulation are injected into 125 mL of dissolution buffer (PBS, 0.01 M, pH 7.4 with sodium azide) prewarmed to 37 C in a 250-mL round bottom flask. The flasks are then agitated at 125 rpm in an orbital shaker. Samples (3 mL) are then removed at 0.25, 0.5, 1, 2, 3, 4, 6, and 24 hr and daily thereafter. The samples are assayed for cerivistatin by high performance liquid chromatography (HPLC). This depot formulation resulted in of drug over a 30-day period.

As another example, a statin depot formulation is prepared by combining 0.5 g of cerivastatin with 9.5 g of a 80:20 mixture of SAIB and ethanol to achieve a homogeneous mixture. The formulation is assayed as described above. Drug release occurs over a 30-day period.

As another example, 1 g of cerivastatin is added to 9 g of a 50:50 mixture of SAIB and benzyl benzoate and mixed by stirring to achieve a homogeneous formulation. Drug release from this formulation occurs over a 60-day period.

In a further example, cerivistatin is added to a solution of poly(lactic acid) (Birmingham Polymers, Inc.) in methylene chloride. The methylene chloride is evaporated and the resulting film is ground to form particles which are added to a mixture of SAIB and N-methyl pyrrolidone (NMP). The final formulation is 45:45:10 SAIB:NMP:poly(lactic acid). The formulation is assayed as described above for statin release. Release occurs over a 60-day period.

Having formulated a SAIB depot it may be injected subcutaneously into the upper are of a subject using a needle and a standard syringe. Alternatively, other parenteral routes of administration may be used. The injection volume and needle size are chosen to optimally achieve the desired rate and duration of release of active agent while minimizing discomfort to the patient. Once the needle is withdrawn, the depot remains under the skin and becomes more viscous as solvent is released from the bulk of the hydrophobic matrix into surrounding tissue. From this stable location, the depot then releases the statin at a relatively steady rate into the surrounding tissue, from where the drug finds its way into the circulatory system, and thence to its site of action. The depot may release the drug for many weeks or months

A biodegradable monolithic rod may also be used. An experimental example of such an embodiment is one in which a monolithic rod is prepared by melt extrusion using a Tinius Olsen extruder, wherein the rod contains 20% statin by weight within a polymer of 65:35 poly (DL-lactide-co-glycolide). The extruded rods are assayed for release of drug by placing in 40 mL of dissolution buffer (PBS, 0.01 M, pH 7.4 with sodium azide) in a 120 or 240-mL amber bottle at 37 C with no agitation. After incubation for 1 hr, 15 mL of buffer is removed for analysis and replaced with fresh buffer. Samples re removed for analysis daily for one week and weekly thereafter. The amount of drug present is determined by HPLC. This formulation releases drug over a 90-day period.

Many modifications may be made to adapt a particular situation, material, process, process step or steps, to the objective, spirit and scope of the present invention. All such modifications are intended to be within the scope of the invention.

What is claimed is:

1. A method for lowering cholesterol in a subject, the method comprising parenterally administering a formulation to said subject, said formulation comprising a cholesterol lowering agent, wherein said formulation is administered from a sustained release drug delivery device implanted in said subject.

2. The method of claim 1, wherein said administering is for a period of at least 24 hours.

3. The method of claim 2, wherein said formulation comprises a drug selected from the group consisting of an HMG CoA reductase inhibitor, an HMG CoA synthase inhibitor, a squalene synthase inhibitor, and a squalene epoxidase inhibitor.

4. The method of claim 2, wherein said drug delivery device is selected from the group consisting of: a non-injectable implant and a depot.

5. The method of claim 2, wherein said drug delivery device comprises a depot.

6. The method of claim 5, wherein said depot comprises sucrose acetate isobutyrate.

7. The method of claim 3, wherein said drug delivery device comprises a depot.

8. The method of claim 7, wherein said depot comprises sucrose acetate isobutyrate.

9. The method of claim 2, wherein said drug delivery device comprises a non-injectable implant.

10. The method of claim 9, wherein said non-injectable implant comprises poly (dl-lactide-co-glycolide).

11. The method of claim 4, wherein said formulation comprises a drug selected from the group consisting of an HMG CoA reductase inhibitor, an HMG CoA synthase inhibitor, a squalene synthase inhibitor, and a squalene epoxidase inhibitor.

12. The method of claim 11, wherein said formulation comprises a statin.

13. A method of treatment of a subject having elevated serum cholesterol levels, the method comprising:
administering a cholesterol lowering agent to a subject, said administering being by systemic delivery from an implanted drug delivery device, for a period of at least one week, at a volume rate of less than about 2 ml/day; whereby serum cholesterol levels are reduced in the subject.

14. The method of claim 13, wherein said cholesterol lowering agent comprises an HMG CoA reductase inhibitor.

15. The method of claim 14, wherein the HMG CoA reductase inhibitor is a statin.

16. An implantable sustained-release dosage form for the lowering cholesterol in a subject, said dosage form comprising a drug delivery device and a cholesterol lowering agent, wherein said a cholesterol lowering agent is released from said drug delivery device, for a period of at least seven days, in an amount sufficient to measurably lower cholesterol in said subject.

17. The dosage form of claim 16, wherein said drug delivery device is selected from the group consisting of: a non-injectable implant and a depot.

19. The dosage form of claim 17, wherein said a cholesterol lowering agent is a statin.

20. The dosage form of claim 19 wherein said statin is selected from the group consisting of: lovastatin, pravastatin, simvastatin, fluvastatin, atorvastatin, cerivastatin, rosuvastatin and combinations thereof.

21. The dosage form of claim 17 wherein the cholesterol lowering agent is selected from the group consisting of: an HMG CoA reductase inhibitor, an HMG CoA synthase inhibitor, a squalene synthase inhibitor, a squalene epoxidase inhibitor, and an antihyperlipoproteinemic agent.

22. The dosage form of claim 17 wherein the dosage form delivers the cholesterol-lowering agent at a rate of about 0.01 micrograms per day to about 20 milligrams per day.

23. The dosage form of claim 17 wherein said cholesterol lowering agent is released at a volume of about 0.001 mL per day to about 1 mL per day.

24. The dosage form of claim 17 wherein said cholesterol lowering agent is released for a period of at least one month.

25. The dosage form of claim 17 wherein the drug delivery devivce is selected from the group consisting of: a hydrogel, a bioerodable implant, a biodegradable implant, a microparticulate suspension, a microsphere and a microcapsule.
